(19) United States
(12) Patent Application Publication
(10) Pub. No.: US 2019/0388759 A1
(43) Pub. Date: Dec. 26, 2019

(54) COMPUTER-IMPLEMENTED METHOD FOR PROVIDING LIVE AND/OR ARCHIVED ANTAGONISTIC SPORTS CLASSES TO REMOTE USERS

(71) Applicant: Fei ZHAO, Markham (CA)

(72) Inventor: Fei ZHAO, Markham (CA)

(21) Appl. No.: 16/015,314

(22) Filed: Jun. 22, 2018

(51) Int. Cl. A63B 69/32; H04N 21/242; H04N 21/6377; H04N 21/236; H04N 21/482; H04N 21/258; A61B 5/024; A61B 5/103; A61B 5/22; A61B 5/00; G09B 5/06; A63B 69/00

(52) U.S. Cl. CPC A63B 69/32; H04N 21/242; H04N 21/6377; H04N 21/23605; H04N 21/482; H04N 21/25866; H04N 21/4753; A61B 5/1036; A61B 5/22; A61B 5/4866; G09B 5/065; A63B 69/004; A61B 5/024

(57) ABSTRACT

The present disclosure relates to a computer-implemented method for providing live and/or archived antagonistic sports classes to remote users. The method comprises providing, by means of a processor, a first user interface associated with a first training terminal of a first user at a first remote location and displaying live and/or archived antagonistic sports classes on the first user interface, so that the first user selects classes therefrom; receiving a selection from the first user at the first remote location and sending the content of the selected class to the first training terminal accordingly. The method further comprises transmitting a performance data of the first user from the first training terminal to a second training terminal of a second user at a second remote location, for a second user to view. The performance data of the first user is detected via a corresponding sensing module and received therefrom. The method can provide remote users with the required antagonistic sports classes at any time at any position, so that they may get professional guidance from instructors.

### FIELD OF THE INVENTION

The present invention relates to intelligent equipment, methods and media for fitness training. In particular, the present invention relates to computer-implemented methods for providing live and/or archived antagonistic sports training classes.

### BACKGROUND OF THE INVENTION

The work and life of modern society have led people to bear more pressure than ever before. Antagonistic sports have become an important sports training for people to exercise their bodies and release their stress. In terms of human nature, human beings like to compete and like to train with an opponent to raise their level instead of mechanical training blindly facing a dummy. Antagonistic sports include various sports that can be used for confrontation and athletics, including but not limited to various fights, various ball games and so on.

Usually, antagonistic sports, such as kickboxing, are professional and dangerous sports, needs an instructor's accurate guidance. Indeed, without instructor's guidance, blind exercise not only leads to the stagnant professional level of people, but also easily leads to sports injuries. Currently, kickboxing instructors usually need to make an appointment with an opponent or a training partner and go to a stationary fitness studio to find their own instructor to attend live kickboxing classes.

All of these class-based exercises can only be performed at specific time at specific places and are expensive, thus many potential kickboxing amateurs are unable to participate in. Especially for those people traveling on business frequently, they have to give up their favorite live kickboxing classes, because they can't get to a specific place at a specific time, or they can only work on dummies at a local gym.

Humans like to socialize and play games in nature, and loneliness and tedium training can easily be discarded early. Popular instructors have a lot of experience in motivating the interests and enthusiasm of training crowd, but their instructing experience can benefit only a few people who attend on-site classes and cannot reach a wide range of remote kickboxing amateurs. The current kickboxing studio also does not provide kickboxing amateurs with a convenient and friendly video voice chat software platform so that kickboxing amateurs can exchange training experiences with each other and organize online/offline activities.

The problems in the training of kickboxing sports are also widely present in other forms in other antagonistic sports.

### SUMMARY OF THE INVENTION

The present disclosure is directed to solve all the above technical problems existing in the prior art. The present disclosure is also directed to provide an antagonistic sports training method and system for cooperation with a sensing module for measuring the user's performance to assess the training effect using the assessment software module and to display the training effect to the user visually and intuitively. The system incorporates multimedia input and output for tutorials for live broadcasting and/or archiving, social networks for video and audio chatting, archiving and networking of user performance metrics for mutual viewing, and a wide range of modalities of man-machine Interactive antagonistic game and interactive antagonistic games among human users.

In a first aspect of the present disclosure, it is directed to a computer-implemented method of providing live and/or archived antagonistic sports classes to remote users, the method comprising:

providing, with a processor, a first user interface associated with a first training terminal of a first user at a first remote location and displaying live and/or archived antagonistic sports classes on the first user interface so that the first user selects classes therefrom;

receiving, with the processor, a selection from the first user at the first remote location and sending the content of the selected class to the first training terminal accordingly; and

transmitting, with the processor, a performance data of the first user from the first training terminal to a second training terminal of a second user at a second remote location for the second user to view, wherein the performance data of the first user is detected via a corresponding sensing module and received therefrom.

In some embodiment, the sensing module can be integrated in a smart wearable device worn by the corresponding user, and/or the first training terminal of the first user and/or the second training terminal of the second user can be integrated in the smart wearable device worn by the corresponding user.

In some embodiment, the method further comprises: assessing, with the first training terminal, the performance data of the first user received from the corresponding sensing module; transmitting, with the processor, the assessment result from the first training terminal to the second training terminal to display on a second user interface associated with the second training terminal.

In some embodiment, the method further comprises: assessing, with the first training terminal, the received performance data of the first user; displaying, with the processor, the assessment result on a second user interface associated with the first training terminal. In some embodiment, the content of the class comprises at least one of information, audio and video contents of the class.

In some embodiment, the performance data includes at least one of a hit strength, a force-bearing area, a hit position, a hit count, a hit acceleration, a hit speed, a hit direction, a hit posture, a hit track, heart rate, standing position, pressure distribution status of legs, accumulated training time.

In some embodiment, the assessment result comprises at least one of the following: hit accuracy, hit posture accuracy, hit beat matching degree, hit score, and calorie burn.

In some embodiment, the method further comprising: displaying, with the processor, a buddy list and status of a buddy associated with the live and/or archived antagonistic sports class on a third user interface of the first training terminal and/or the second training terminal.

In some embodiment, both of the first user and the second user include plurality of users, and the method further comprises: displaying, with the processor, the comparison between the performance data and/or assessment results of the plurality of first users on a fourth user interface of each user of the plurality of first users and the second users.

In some embodiment, the content of the selected class sent to the first training terminal is fluidized to be displayed to the first user substantially in real time.

In some embodiment, the content of the selected class sent to the first training terminal is the archived content provided by a database.

In some embodiment, the method further comprising: transmitting, with the processor, audio or video chat data from a server or a user terminal of a user to another training terminal of another user, so as to display the audio or video chat data to the user on the user interface of the another training terminal of the another user.

In some embodiment, the method further comprises: transmitting, with the processor, details of the performance data or assessment result from the first training terminal to the second training terminal, so as to display the details on a second user interface associated with the second training terminal.

In some embodiment, the method further comprises: displaying, with the processor, details of the performance data or assessment result from the first training terminal on the second user interface associated with the first terminal.

In some embodiment, the antagonistic sports of the antagonistic sports classes comprise any one of boxing, kickboxing and mixed martial arts.

In a second aspect of the present disclosure, it is directed to a computer storage medium with computer-executable instructions stored thereon, which performs the following steps when executed by a processor:
receiving performance data of antagonistic sports training from a sensing module;

processing based on the performance data to obtain at least one of a performance metric and an assessment result;

transmitting at least one of the obtained performance metric and the assessment result to a display for displaying.

In some embodiment, computer-executable instructions, when executed by the processor, further perform the following steps: transmitting at least one of the performance metrics and assessment result to a server or another user terminal.

In some embodiment, the computer-executable instructions, when executed by the processor, further perform the following steps: providing a first user interface for a first training terminal of a first user at a first remote location and displaying live and/or archived antagonistic sports classes on the first user interface for the first user to select therefrom;
receiving information of the live and/or archived antagonistic sports classes and audio and video content of the selected class from the server or another user terminal and displaying the same on the second user interface of the first training terminal of the first user.

In some embodiment, the performance data comprises at least one of hit strength, force-bearing area, hit position, hit count, hit acceleration, hit speed, hit direction, hit posture, a hit track, heart rate, standing position, pressure distribution status of legs, the accumulated training time.

In some embodiment, the assessment result includes at least one of: hit accuracy, hit posture accuracy, hit beat matching degree, hit score, and the burned calorie.

In some embodiment, the computer-executable instructions, when executed by a processor, further perform the following steps: providing the first training terminal of the first user at the first remote location with a third user interface, and displaying a buddy list and status of a buddy associated with the live and/or archived antagonistic sports class on the third user interface.

In some embodiment, the computer executable instructions, when executed by the processor, further perform the steps of: receiving at least one of the performance metric and the assessment result from a second user terminal of the second user at the second remote location; displaying a comparison between at least one of the performance data and assessment result of the first user and the second user on the third user interface of the first user terminal of the first user.

### BRIEF DESCRIPTION OF THE DRAWINGS

Features, advantages, and technical and industrial significance of exemplary embodiments of the invention will be described below with reference to the accompanying drawings, in which like reference numerals denote like elements, it is to be noticed that the terms “first” and “second”, as used herein, are used as for distinction only and are not intended to limit number, for example, “first user,” “first training terminal,” and “A intelligent wearable device” may represent a plurality of users, a plurality of training terminals and a plurality of intelligent wearable devices, when referring to the “first user interface” of different training terminals, a similar or functionally similar user interface may be indicated, which does not necessarily represent the same user interface, and wherein:

FIG. 1 illustrates a diagram of a standalone kickboxing training system for cooperation with the intelligent wearable device according to a first embodiment of the present disclosure;

FIG. 2 illustrates a diagram of a networked kickboxing training system for cooperation with the intelligent wearable device according to a second embodiment of the present disclosure;

FIG. 3 illustrates a flowchart of content generation and distribution according to a third embodiment of the present disclosure;

FIG. 4 illustrates a diagram of a first user interface displayed on a user terminal according to a fourth embodiment of the present disclosure;

FIG. 5 illustrates a diagram of a user interface providing a kickboxing class schedule displayed on a user terminal according to a fifth embodiment of the present disclosure;

FIG. 6 illustrates a diagram of a user interface displaying live or non-real-time classes in progress on a user terminal according to a sixth embodiment of the present disclosure; and

FIG. 7 illustrates a diagram of a user interface displaying a trainee's training performance and other information on an instructor's terminal according to a seventh embodiment of the present disclosure.

### DETAILED DESCRIPTION OF THE INVENTION

The following description is intended to enable persons skilled in the art to understand and practice the present invention. The following description of specific embodiments or applications is provided by way of example only. Various modifications to the various embodiments will be readily apparent to those skilled in the art, and the main principles defined herein may be applied to other embodiments and applications without departing from the spirit and scope of the invention. Therefore, the invention is not intended to be limited to the particular embodiments shown, but rather to claim the scope limited only by the claims, in accordance with the principles and characteristics disclosed by this invention. Although the respective embodiments are described respectively, the features and elements in the different embodiments may be combined with each other, and the combination manner is not limited to each independent embodiment.

The present disclosure is directed to provide a computer-implemented method for providing live and/or archived antagonistic sports classes to remote users. Antagonistic sports include various sports that can be used for confrontation and athletics, including but not limited to various fights, various ball games and so on, Hereinafter, taking the kickboxing as an example of antagonistic sports, those skilled in the art can easily apply the various embodiments disclosed below to other antagonistic sports, and in particular can be easily applied to any one of boxing and mixed martial arts, can also be easily applied to a specialized kickboxing sports, including but not limited to, karate, taekwondo, Jeet Kune Do, Tai Chi, etc., which will not be described here for concise.

Stand-Alone Kickboxing Training System

FIG. 1 illustrates an on-site kickboxing training system for use with a sensing module 100 cooperatively in accordance with a first embodiment of the present disclosure. The sensing module 100 is configured to measure the performance of a user's on-site kickboxing sports. The sensing module 100 can take various forms, for example, a set of sensing modules 100 mounted on a dummy, or a sensing module 100 worn on a user, for example, it can be installed in the user's kickboxing protective gears (including kickboxing gloves), it can also be worn on the user's body (such as smart bracelet) and so on. In some embodiments, the sensing module 100 may be integrated in a smart wearable device (for example, smart bracelet) worn by the corresponding user.

As shown in FIG. 1, the on-site kickboxing training system can cooperate with various forms of sensing modules 100, and may include: a user terminal 106 communicably connected to the sensing module 100 via the communication port 110 to receive the measured performance data therefrom and transmitting display information to the display 108 via the communication port 110 on the basis of the received performance data; and the display 108 performing display based on the display information received from the user terminal 106.

The display 108 can be installed anywhere within the user's visual range. And, the user terminal 106 may be driven by a user input device such as a touch screen, a mouse, a joystick, a remote controller, or other devices. The display 108 can be a variety of external displays, including mobile phones, tablets, desktops, TVs, projectors and the like.

The user terminal 106 may, for example, include: a communications interface 110 for receiving and transmitting information; a processor 105; an audio/video module 112 for playing music and/or video in accordance with control instructions; and a memory 107 in which an assessment module 109 and class module 113 (113a-113n) are stored, when the class modules 113 are performed by the processor 105, the controlling instruction may be sent to the corresponding device so that it can be operated according to the class settings.

The processor 105 may be a processing device including one or more general-purpose processing devices such as a microprocessor, a central processing unit (CPU), a graphics processing unit (GPU), and the like. More specifically, the processor 105 may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. Processor 105 may also be one or more specialized processing devices such as application specific integrated circuits (ASICs), field programmable gate arrays (FPGAs), digital signal processors (DSPs), system on chip (SoCs) and the like. The processor 105 may be communicatively coupled to the intelligent wearable device integrated with the sensing module 100 via the communication port 110, and may also be integrated into the intelligent wearable device.

The user terminal 106 including the processor 105 may be associated with the intelligent wearable device, and may be, for example, a local computer, a remote server, a cloud-based server, or may be integrated into the intelligent wearable device. Preferably, the user terminal 106 is separated from the intelligent wearable device thereby avoiding the impact of hit and vibration on its operation performance while allowing the intelligent wearable device to retain only the necessary components, making it light and compact and less prone to be damaged and easier to carry.

The user terminal 106 may include a memory 107 for storing performance data for a plurality of users performing kickboxing training using the sensing module 100, so as for user to recall and query. The memory 107 may store computer-executable instructions, when the computer-executable instructions is executed by the processor 105, the following step may be performed: controlling the related device to present performance data; assessing a user's hit action to display the assessment result to a user; controlling the related device such as a display, a video camera, a microphone, a speaker, or other audio output device, and the like to perform the corresponding operation.

The user's hit action may be assessed based on the performance data of the hit made by the user that is measured by sensing module 100 to display the assessment result to the user. For example, the accuracy of the user's hit location may be assessed based on the stress bearing area and the hit location of the user's hit; the user's hit intensity may be assessed based on the user's real-time hit speed and hit strength; or whether the user's hit posture is correct and whether the hit action is in place may be assessed based on the user's hit direction, and so on. Optionally, the assessment module 109 may further include a scoring system to score the hit action according to at least one of the user's hit position, hit direction, hit intensity, hit posture and hit track. Introducing music rhythms into kickboxing classes can increase the fun of kickboxing training and enhance the enthusiasm of trainees. Trainees can punch and kick following the beat of the music so that their body coordination can be exercised in all dimensions. Optionally, the assessment module 109 may assess the degree of beat matching of the user's hit action based on the beat of the music and the user's hit time.

For example, the class schedule may be displayed on the display 108, allowing the user to select class g to participate via the input device. The processor 105 is configured to: recall and execute the corresponding class module 113g based on the received selection of the class by the user, and send the control instruction to the corresponding devices. For example, the processor 105 is configured to send a control instruction for audio playing to the audio/video module 112 to make it play music according to the timing of the class g, and send a control instruction for illuminating the light source to the light source on the dummy to provide a prompt, in accordance with the setting of class g, to users on the hit locations at specific times. While the user kickboxing the dummy, the performance data collected by the sensing module 100 is transmitted to the user terminal 106 approximately in real time. The processor 105 is configured to process the received performance data, for example, to process the performance data as an easy-to-understand performance metric, e.g., to convert the collected hit acceleration to hit speed, or to assess the performance data to get an assessment result, then transmit the corresponding display information to the display 108 for corresponding display, for example, the first round: the hit strength is 50 kg, the hit count is 15, the hit speed is 50 m/s, the hit accuracy is 8/10, hit beat matching degree is 7/10, and so on. In this document, the term “performance data” is data of the user's performance obtained by using the sensing module 100. The term “performance metric” is an easily understood parameter obtained by further processing the performance data obtained by the sensing module 100. According to different functions of the sensing module 100, the “performance metric” corresponding to one sensor module 100 may also be the “performance data” directly obtained by another sensor module 100.

In some embodiments, the performance data may include the movement track and the hit area detected by the sensor. The corresponding performance metric may include the types, numbers, and combinations (formats) of the various hit actions derived from the two. The various hit actions include but are not limited to jabs, crosses, hooks, uppercuts, front kicks, sidekicks, roundhouse kicks and so on.

In addition, the remaining time for this round can also be displayed on the display 108. Further, the user's position on the leaderboard (top 20%) and this round's score may be displayed on the display 108 as well as the calories burnt by the user for this round, and so forth.

Please note that the sensing module 100 may include a sensing device outside the dummy and user to collect richer performance data. For example, the external sensing device may include: a camera 114a for capturing the position and posture of the user; a blanket 114b on which a sensor is provided for sensing the standing position of the user's both feet, the pressure distribution condition of the both legs, and the like. Using the performance data measured by both of them, it is possible to determine the center of gravity of the user, the distance to the punching bag, the height of the center of gravity, the posture, and so on, which can be used to further assess whether the user's kickboxing action is correct or not, and can also provide visual tips on the display 108 or audio output tips for improving kickboxing action: for example, to change the foot on which the center of gravity is located, move 0.5 meters towards the sandbag, lower the center of gravity by 20 cm and so on.

Optionally, the system is equipped with a speaker 111 to create an immersive kickboxing sound scene and improve the user's training experience. Instead of the speaker 111, a wireless headset (for example a Bluetooth headset) may be provided to be worn by the user and communicably connected with the audio/video module 112 of the user terminal 106, so as to reduce the interference with the outside world meanwhile creating the sound scene for the user, thereby expanding the training space so as that users can participate in training anytime, anywhere.

The above user terminal 106 may be any one of a mobile phone, a portable intelligent assistant, a tablet computer and a desktop computer provided that the communication port 110, the audio/video module 112, the processor 105 and the memory 107 are equipped. The various modules including the assessment module 109 and the class module 113 may be operated by using a local operating system or application of the local operating system such as Android or IOS application or via a browser-based system.

Networked Kickboxing Training System

The above stand-alone kickboxing training system can be expanded to networked kickboxing training system through networking.

In the networked state, the applications in the user terminal 106 may be used to display various user interfaces, the user terminal 106 may be connected to the back-end server using any suitable network, such as the Internet, and the control and calculation of various assessments and class may be executed by the user terminal 106 or may be executed by the back-end server. In some embodiments, the back-end server of the center may also be canceled and the corresponding operations may be executed in distributed or peer-to-peer manner instead.

In various exemplary embodiments, a networked training system may allow users to create accounts and save and manage their performance data. The system can allow users to browse upcoming live broadcast schedules, register for future live broadcast classes, and set reminders. Users can also invite others to live broadcast classes and set text, email, voice or other notifications and calendar entries. In addition to the user interface of a local system (stand-alone system) associated with his intelligent wearable device 100, a user may be able to access the system, account, performance data, or all other data via a web-based or application-based interface for desktops and/mobile devices.

In various exemplary embodiments, the system may allow multiple users to participate in the scheduled class simultaneously, which are synchronized by the system, and allow access to all the same communication and data sharing features available for the live broadcast class. With this feature, users (kick boxers) participating in the same class can compete with each other and with past performance of themself or “ghost” users in the same class.

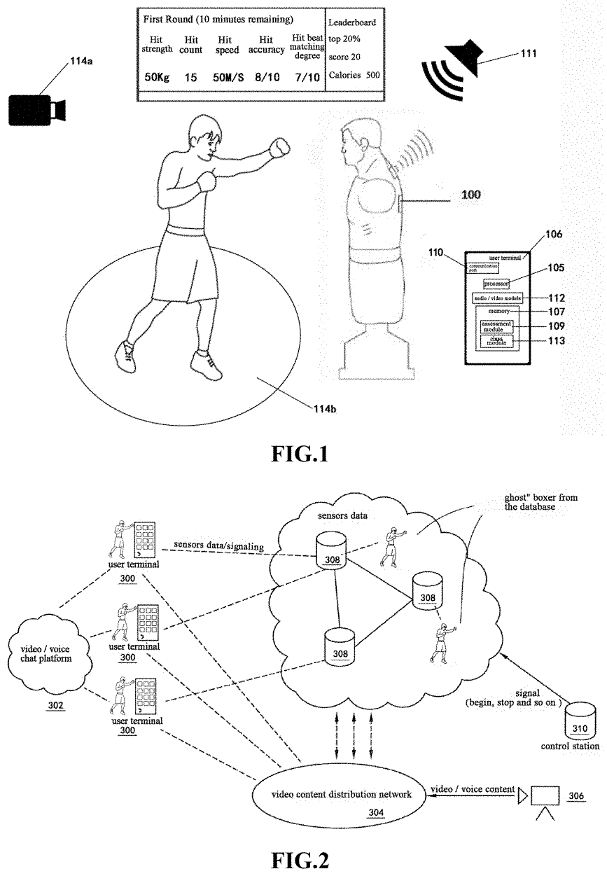

FIG. 2 illustrates a networked kickboxing training system for use with the sensing module cooperatively according to a second embodiment of the present disclosure. The system may be configured to feed the user with synchronized live and/or archived video content and live and/or archived sensor data over the network. In various exemplary embodiments, a networked kickboxing training system may be configured with: a plurality of user terminals 300 in communication with the video/voice chat platform 302; and a video content distribution network 304 that receives audio video content from one or more content sources 306. The user terminal 300 may also communicate with various other networks and servers. For example, user terminal 300 may exchange sensors and render data and/or signaling with various databases 308, including historical or “ghost” data. The control station 310 may provide signals over the network to control the collection, storage and management of data in the system.

Content Generation and Distribution

In the kickboxing training system described above, kickboxing-training video content needs to be generated and uploaded and distributed to users, allowing users to autonomously select favorite video content to watch and learn and generate their own training video for uploading and sharing with friends.

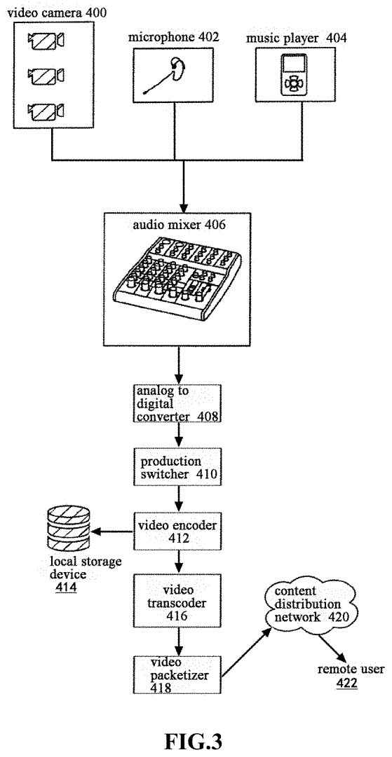

FIG. 3 shows a flowchart of content generation and distribution according to the third embodiment of the present disclosure. Content distributed over the network can be created in a variety of different manners. In various exemplary embodiments, recording may be conducted at an instructor-led kickboxing training session with trainee-live participation or may be conducted in dedicated studios with no live, in-studio participation or may be flexibly completed by the user with the video capture capabilities of the smartphones during training. Recording equipment including a microphone and one or more cameras may be used to capture instructors and/or trainees during the class. Multiple cameras can provide different views, and 3D cameras can be used to create 3D content. In various exemplary embodiments, users may also generate live or recorded classes locally by using local cameras and microphones (for example intelligent phones and intelligent tablet computers that integrate these capabilities) that can be transferred and stored on the system, and distributed throughout the network.

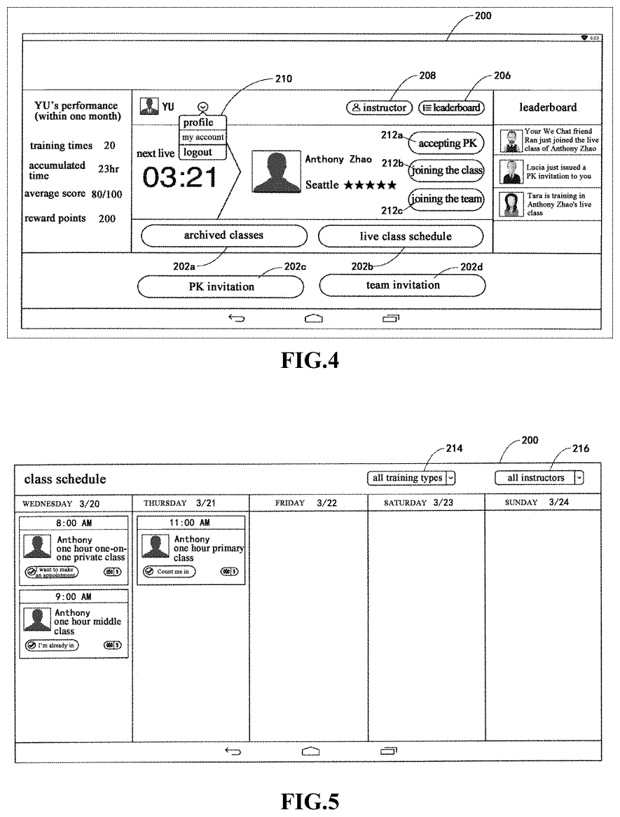

As shown in FIG. 4, one or more video cameras 400, a microphone 402, and a music player 404 may be used as inputs to the audio mixer 406 to generate class content. The audio mixer 406 outputs the content to the analog to digital converter 408, which provides the converted data to the production switcher 410 to send the produced video to the video encoder 412. Video encoder 412 stores the encoded video to a local storage device 414 and sends it to a video transcoder 416. The video transcoder 416 outputs the transcoded data to a video packetizer 418 and then sends the packetized data stream to the remote user 422 through the content distribution network 420. The packetized data streams may also be transmitted to the server as various training classes for remote users 422 to access and to the corresponding remote users 422 via the content distribution network 420.

User Interface

In various exemplary embodiments, a user may use a touch screen or other interface to selectively present a series of different information including live and/or archived video, performance data, and other users and system information on the screen. The user interface can provide a wide range of control and information windows that can be accessed and removed individually and/or in groups by clicking, touching or gesturing. In various exemplary embodiments, such windows may provide information about the user's own performance and/or the performance of other participants in the same class in the past and/or at present.

The user interface can be used to access member information, login and logout of the system, access live content such as live exercise classes and archived content (referred to in the Figures as “non-real time classes”). User information may be displayed in a variety of formats and may include historical and current performance and account information, social networking links and information, reward points, etc. The user interface can also be used to access the system to update profile or member information, manage account settings such as information sharing, and control device settings.

The user interface 200 can be smoothly switched between the interfaces of different contents under the user's input, and these different interfaces use the reference numeral 200 for convenience of description.

FIG. 4 illustrates a diagram of one example of a home interface displayed on a first user terminal of a first user at a first remote location according to a fourth embodiment of the present disclosure. As shown in FIG. 4, such a main interface may provide direct links to information such as scheduled classes 202b, archived classes 202a, a leaderboard 206, instructor 208, and/or profile and account information 210. The main interface may display user Yu's kickboxing performance over a specified time period, for example, including the number of training sessions, the accumulated time, the average score, the points, and the like. By pulling down and selecting items from the instructor menu 208, the instructor Anthony Zhao's photo, address, rating level (e.g. five-star rating), and information for the next live class can be displayed on the main interface. On a side of the main interface, the dynamic information of other users related to the user can be scrolled, for example, “Your We Chat friend Ran just joined the live class of Anthony Zhao”, “Lucia just issued a PK invitation to you” and “Tara is training in Anthony Zhao's live class.” The main interface also provides a number of function keys, including accepting PK (212a), joining the class (212b), joining the team (212c), PK invitation (202c), team invitation (202d), etc., so that the user may complete the corresponding function upon selecting and clicking corresponding function keys. For example, when a user sees a dynamic message “Lucia just sent you a PK invitation,” he/she may enter the PK mode with Lucia by clicking on Lucia's avatar and clicking the “Accepting PK” function key.

In various exemplary embodiments, a first user interface associated with the first user terminal of the first user at a first remote location may be provided and live and/or archived antagonistic sport classes may be displayed on the first user interface for the first user to make a selection out of the live and/or archived content. In some embodiments, for example, if the user selects live class table 202b, they may be presented with a display interface that displays the schedule of upcoming classes.

FIG. 5 shows an exemplary schedule of upcoming classes presented through the user interface 200, where the classes are displayed in a traditional calendar manner. Pull-down or other display features allow the user to find classes by training type 214, instructor 216, or any other appropriate category. The schedule can be presented in any suitable format, including calendars, lists and thumbnails and so on. Optionally, the presentation format can be autonomously selected by users. In addition to the class information, such as 1 hour private class, 1 hour primary class, etc., other information may also be displayed on the respect class items on the schedule, including the state of the user related to the class and the function keys to be selected by the user, for example, the status includes “I am already in”, “Already Full”, function keys include “I want to make an appointment”, “Count me in” and so on. Optionally, the curriculum schedule of the user interface 200 may display the classes that may be started in the future for users to reserve through the function keys, and automatically notify the instructor to start the class when the reservation reaches the threshold.

In various examples, if the user selects the archived class 202a, the archived classes may be presented on the user interface in a variety of ways, including but not limited to, list, icon, thumbnail, Scroll broadcast, etc. . . .

FIG. 6 shows a main window 220 showing the content of live or archived classes selected by the user. In various exemplary embodiments, the performance metrics and/or assessment results windows 222, 224, 226, 228, and 230 may display specific performance metrics and/or assessment results for the user's current training, past training, or other performance information. Such performance metrics and/or assessment results windows may be presented anywhere on the display screen and may be selected by users such that they may be displayed or removed by a screen touch or gesture. As shown in FIG. 6, window 222 shows the hit count and the hit rate (the ratio of the count of scored hits/the hit count). The window 224 shows the user's hit strength, the user's average hit strength and maximum hit strength, and an indicator arrow 232 indicating whether the user's hit strength is better than or worse than the average performance. The window 226 shows the user's score in this class period, the average and maximum score of the individual periods of this class, and the class average score in this class period. It should be noted that the score may be calculated by an assessment rule set by the system, for example, the score may be assessed by the assessment module 109 residing on a server or a user terminal as described above. The assessment rule may strictly follow the rules of the kickboxing match, or it may be customized specific for the needs of users. For example, for the classes respectively aimed at improving the hit strength and correcting the kickboxing posture, the weights of hit strength and the accuracy of the kickboxing posture are increased respectively when calculating the scores. The assessment rules can be set independently by the instructor or pre-set by the system. Window 228 shows the degree of beat matching between the user's hit and the music beat, while window 230 shows the burned calories and the heart rate. Note that the performance metrics and/or assessment results windows listed here are only examples, and a variety of other performance metrics and/or assessment results can also be displayed.

In some embodiments, the first user's performance data may be transmitted from the first user terminal to the second user terminal of the second user at the second remote location, for the second user to view. For example, the second user can recall and view the first user's performance data. In some embodiments, the first user's performance data may be displayed on the second user's display interface in the manner of the leaderboard 234.

As an improvement to this embodiment, leaderboards 234 may also be displayed to allow the user to see how their performance in comparison to others in the same class. In various exemplary embodiments, the leaderboard may be configured to display the relative performance of all trainees or one or more groups of trainees. For example, the user may select a leaderboard that displays the performance of trainees in a particular age group, male trainees, female trainees, male trainees in a particular age group, trainees in a particular geographic area, etc. Users can move the leaderboards on the interface cancel the leaderboards and remove them from the interface with gestures and buttons. In various exemplary embodiments, the system may incorporate various social networking aspects, such as allowing users to fan other users or to create user groups or circles. User lists and information may be accessed, categorized, filtered, and used in a wide range of different ways. For example, other users may be classified and/or grouped based on any characteristic including personal information such as age, gender, weight, or based on performance (for example, current score, speed of hit, hit count, or a custom score).

Although the leaderboard 234 in FIG. 6 shows the total scores of different users, this is merely an example. The leaderboard 234 may also display other performance metrics and/or assessment results at different times of the same user, for example, hit beat matching, so as to enable the user to perform a horizontal comparison of various performance metrics and/or assessment results of the user to perform more targeted training.

In the following, the description of the live courses is given with the instructor as an example of the second user and the remote trainee as the example of the first user, but it should be noted that the first user and the second user are not limited to this setting and may also be other persons.

In live classes, instructors can instruct remote trainees to make kickboxing actions with background music, such as giving a “punch” instruction at a music beat and providing targeted guidance based on a student's voice feedback; remote trainees can take advantage of the local display interface 200 as shown in FIG. 6 to watch and learn from live classes and see their own specific performance. Optionally, the instructor-end display interface 200 may display a list of trainees who follow the instructor in the live class and enable the instructor to select each trainee via the display interface 200 to look over the performance and assessment of the corresponding trainee, thereby providing targeted guidance. The instructor-end display interface 200 may display the main performance data and assessment result of all the trainees in the form of thumbnails 254. Optionally, the performance data includes at least one of the following: hit strength, force-bearing area, hit position, hit count, hit acceleration, hit speed, hit direction, hit posture, hit track, heart rate, standing position, pressure distribution status of legs, the accumulated training time. The evaluation result includes at least one of the following: hit accuracy, hit posture accuracy, hit beat matching degree, hit score, and the burned calorie. For example, an instructor can let Jane to take a break upon seeing Jane's heart rate suddenly rise to 180 BPM, instruct Lucia to punch following the beats upon seeing Lucia's hit beat matching degree is low and so on.

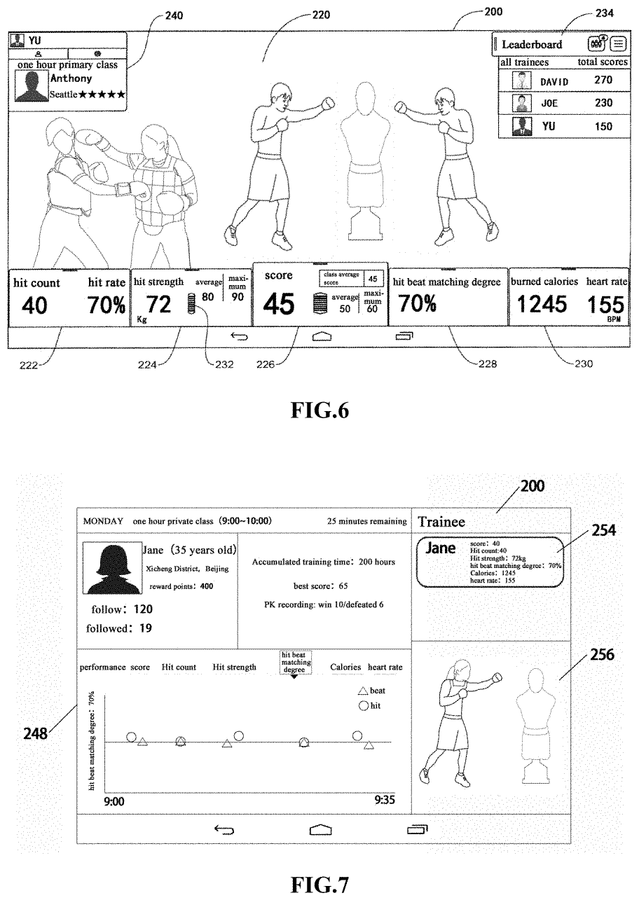

In a case that an instructor needs to know more about a trainee's kickboxing performance details to provide targeted guidance, for example, when offering one-on-one private classes, the instructor may click Jane's thumbnail 254 to show details of her performance and other information as shown in FIG. 7. At the top of the user interface 200 shown in FIG. 7, specific information and remaining time of the class period are displayed; personal information of the trainee Jane is displayed below the class information so that the instructor can better communicate with her and provide appropriate instruction. In the lower left window 248, a pull-down menu of trainee Jane's score, hit speed, hit count, hit beat matching degree, calories, heart rate and the like is displayed. In addition to displaying the values in the thumbnails, the pull-down menu will show more performance details. Taking hit beat matching degree as an example, after pulling down the hit beat matching degree menu, the beat time point and the hit time point will be displayed during the full time period from 9:00 to 9:35. As shown in window 248, there are a total of five beat time points (represented by triangles) during the full time period of 9:00 to 9:35, and the trainee Jane's hit times coincide with the second and fourth beat time points better, but earlier with respect to the first and fifth beat time points, and later with respect to the third beat time point. Upon grasping these performance details, the instructor can provide trainees with more detailed and effective guidance.

If necessary, the instructor may choose to display a window of trainee Jane's live training in the user interface 200 to look over her posture and performance.

The various embodiments described above are merely examples of the solutions of the present disclosure, and the solutions of the present disclosure may be implemented with fewer features than the respective embodiments, and the features of the respective embodiments may be combined with each other.

Various operations or functions are described herein, which may be implemented as software code or instructions or defined as software code or instructions. Such content may be source code or differential code (“delta” or “patch” code) that can be executed directly (“object” or “executable” form). The software implementation of the embodiments described herein may be provided via an article of manufacture storing code or instructions therein or via a method in which a communication interface is operated to send data via the communication interface. The machine or computer readable storage medium may cause the machine to perform the described functions or operations, and include any mechanism for storing information in the form accessible by a machine (e.g., computing device, electronic system, etc.), such as recordable/non-recordable media (e.g., read-only memory (ROM), random access memory (RAM), disk storage media, optical storage media, flash memory devices, etc.). The communication interface includes any mechanism that interfaces with any of the hardwired, wireless, optical mediums and the like, to communicate with another device, such as a memory bus interface, a processor bus interface, an Internet connection, a disk controller, and the like. By providing configuration parameters and/or sending a signal, the communication interface may be configured to prepare the communication interface to provide a data signal describing the software content. The communication interface may be accessed via one or more commands or signals sent to the communication interface.

The invention also relates to a system for performing the operations herein. The system may be constructed specifically for the intended purpose, or the system may include a general-purpose computer that is selectively activated or reconfigured by a computer program stored in the computer. Such a computer program may be stored in a computer readable storage medium such as, but not limited to, any type of disk including a floppy disk, an optical disk, a CDROM, a magneto-optical disk, a read only memory (ROM), a random-access memory (RAM), an EPROM, an EEPROM, a magnetic card or an optical card, or any type of medium suitable for storing electronic instructions, wherein each medium is coupled to a computer system bus.

The above examples are only exemplary embodiments of the present invention and are not intended to limit the scope of the invention, which is defined by the claims. Those skilled in the art may make various modifications and equivalent replacements to the invention within the essence and protection scope thereof, and such modifications and replacements may be regarded as falling in the protection scope of the invention.

1. A computer-implemented method of providing live and/or archived antagonistic sports classes to remote users, the method comprising:
providing, with a processor, a first user interface associated with a first training terminal of a first user at a first remote location and displaying live and/or archived antagonistic sports classes on the first user interface so that the first user selects classes therefrom;
receiving, with the processor, a selection from the first user at the first remote location and sending the content of the selected class to the first training terminal accordingly; and
transmitting, with the processor, a performance data of the first user from the first training terminal to a second training terminal of a second user at a second remote location for the second user to view, wherein the performance data of the first user is detected via a corresponding sensing module and received therefrom.

2. The method of claim 1, wherein, the sensing module can be integrated in a smart wearable device worn by the corresponding user, and/or
the first training terminal of the first user and/or the second training terminal of the second user can be integrated in the smart wearable device worn by the corresponding user.

3. The method of claim 1, wherein, the method further comprises:
assessing, with the first training terminal, the performance data of the first user received from the corresponding sensing module;
transmitting, with the processor, the assessment result from the first training terminal to the second training terminal to display on a second user interface associated with the second training terminal.

4. The method of claim 1, wherein the method further comprises:
assessing, with the first training terminal, the received performance data of the first user;
displaying, with the processor, the assessment result on a second user interface associated with the first training terminal.

5. The method of claim 1, wherein that the content of the class comprises at least one of information, audio and video contents of the class.

6. The method of claim 1, wherein the performance data includes at least one of a hit strength, a force-bearing area, a hit position, a hit count, a hit acceleration, a hit speed, a hit direction, a hit posture, a hit track, heart rate, standing position, pressure distribution status of legs, accumulated training time.

7. The method of claim 3, wherein the assessment result comprises at least one of the following: hit accuracy, hit posture accuracy, hit beat matching degree, hit score, and calorie burn.

8. The method of claim 1, wherein the method further comprising: displaying, with the processor, a buddy list and status of a buddy associated with the live and/or archived antagonistic sports class on a third user interface of the first training terminal and/or the second training terminal.

9. The method of claim 1, wherein, both of the first user and the second user include plurality of users, and the method further comprises: displaying, with the processor, the comparison between the performance data and/or assessment results of the plurality of first users on a fourth user interface of each user of the plurality of first users and the second users.

10. The method of claim 1, wherein the content of the selected class sent to the first training terminal is fluidized to be displayed to the first user substantially in real time.

11. The method of claim 1, wherein the content of the selected class sent to the first training terminal is the archived content provided by a database.

12. The method of claim 11, wherein, the method further comprising: transmitting, with the processor, audio or video chat data from a server or a user terminal of a user to another training terminal of another user, so as to display the audio or video chat data to the user on the user interface of the another training terminal of the another user.

13. The method of claim 3, wherein, the method further comprises:
transmitting, with the processor, details of the performance data or assessment result from the first training terminal to the second training terminal, so as to display the details on a second user interface associated with the second training terminal.

14. The method of claim 4, wherein, the method further comprises:
displaying, with the processor, details of the performance data or assessment result from the first training terminal on the second user interface associated with the first terminal.

15. The method of claim 1, wherein, the antagonistic sports of the antagonistic sports classes comprise any one of boxing, kickboxing and mixed martial arts.

16. A computer storage medium with computer-executable instructions stored thereon, which performs the following steps when executed by a processor:
receiving performance data of antagonistic sports training from a sensing module;
processing based on the performance data to obtain at least one of a performance metric and an assessment result;
transmitting at least one of the obtained performance metric and the assessment result to a display for displaying.

17. The computer storage medium of claim 16, wherein, computer-executable instructions, when executed by the processor, further perform the following steps:
transmitting at least one of the performance metrics and assessment result to a server or another user terminal.

18. The computer storage medium of claim 16, wherein, the computer-executable instructions, when executed by the processor, further perform the following steps:
providing a first user interface for a first training terminal of a first user at a first remote location and displaying live and/or archived antagonistic sports classes on the first user interface for the first user to select therefrom;
receiving information of the live and/or archived antagonistic sports classes and audio and video content of the selected class from the server or another user terminal and displaying the same on the second user interface of the first training terminal of the first user.

19. The computer storage medium of claim 16, wherein, the performance data comprises at least one of hit strength, force-bearing area, hit position, hit count, hit acceleration, hit speed, hit direction, hit posture, a hit track, heart rate, standing position, pressure distribution status of legs, the accumulated training time, and
the assessment result includes at least one of: hit accuracy, hit posture accuracy, hit beat matching degree, hit score, and the burned calorie.

20. The computer storage medium of claim 16, wherein, the computer-executable instructions, when executed by a processor, further perform the following steps:
providing the first training terminal of the first user at the first remote location with a third user interface, and displaying a buddy list and status of a buddy associated with the live and/or archived antagonistic sports class on the third user interface,
wherein, the computer executable instructions, when executed by the processor, further perform the steps of:
receiving at least one of the performance metric and the assessment result from a second user terminal of the second user at the second remote location;
displaying a comparison between at least one of the performance data and assessment result of the first user and the second user on the third user interface of the first user terminal of the first user.
